(19) United States
(12) Patent Application Publication
(10) Pub. No.: US 2022/0347083 A1
(43) Pub. Date: Nov. 3, 2022

(54) COSMETIC COMPOSITIONS COMPRISING MELANOIDINS

(71) Applicant: Kaffe Bueno ApS, Soborg (DK)

(72) Inventors: Juan Pablo MEDINA, Copenhagen (DK); Adnan Akin AKÇAY, København (DK)

(73) Assignee: Kaffe Bueno ApS, Soborg (DK)

(21) Appl. No.: 17/757,741

(22) PCT Filed: Dec. 23, 2020
(86) PCT No.: PCT/EP2020/087838

§ 371 Date: Jun. 20, 2022

(30) Foreign Application Priority Data
Dec. 23, 2019 (EP) 19020723.3

(51) Int. Cl. A61K 8/9789; A61Q 5/06; A61Q 1/02

(52) U.S. Cl. CPC A61K 8/9789; A61Q 5/065; A61Q 1/02; A61K 2800/30; A61K 2800/805; A61K 2800/43

(57) ABSTRACT

The present application is directed to a cosmetic composition comprising melanoidins, wherein said melanoidins are characterized by having a weight average molecular weight (Mw) of from 200 daltons to 300 kDa, preferably from 1 to 300 kDa and more preferably from 1 to 100 kDa, for example from 10 to 100 kDa. The melanoidins are obtainable or are obtained by a process comprising the steps of: a) providing a particulate foodstuff which comprises melanoidins; b) treating said particulate foodstuff with an aqueous extractant solution having a pH greater than 7 to extract melanoidins as solute in the aqueous phase; c) physically separating said aqueous phase from the residual treated particulate foodstuff; d) fractionating the dispersed or dissolved components of said aqueous phase on the basis of molecular weight; and, e) dehydrating said fractionated aqueous phase to obtain at least one solid melanoidin fraction therefrom.

### FIELD OF INVENTION

The present invention is related to the use of melanoidins in cosmetics. More particularly, it is directed to the use in cosmetics of melanoidins defined by a molecular weight range, which melanoidins are extractable from primary sources and from waste sources such as spent coffee grounds.

### BACKGROUND TO THE INVENTION

Melanoidins are a class of brown, hydrophilic nitrogen-containing polymers which are formed during the thermal processing of foods—such as coffee, cocoa, bread, malt, barley, Brewers Spent Grain (BSG), soy, meat, and honey—and they are, as such, widely distributed in the human diet. During thermal processing of such foods, amino acids and reducing sugars, such as aldose and d-xylose, react to form what are termed initial Maillard reaction products. Under continued heating, melanoidins are formed by cyclizations, dehydrations, retro-aldolizations, rearrangements, isomerizations and condensations of those initial Maillard reaction products. The complexity of the Maillard reaction pathways results in a range of final reaction products, with inter alia the time of heating, type of heating, temperature, initial chemical composition of the system, moisture content, water activity and pH being determinative of the final composition.

By way of example, the roasting process of the green coffee bean may be represented as a two-stage transformation: i) the evaporation of free water from the bean; and, ii) pyrolysis within the beans, with concomitant swelling and darkening of the beans and loss of dry weight. Oligosaccharides, polysaccharides and proteins present in the green coffee beans degrade and may then participate in the Maillard reactions. There is consequently a significant change in the chemical composition of the beans during roasting with melanoidins being a predominant product but caffeine and chlorogenic acids also being present in the final roasted coffee bean.

An analysis of the art reveals that significant attention has been given to melanoidins in coffee. Certainly, coffee is a food product that is relatively rich in melanoidins, with levels as high as 28 wt. %. Coffee brew melanoidins are water-soluble—rendering research less complicated—and, further, coffee is of great commercial importance given that it is consumed by millions of people worldwide every day. It is here stated that, whilst coffee melanoidins do represent a preferred focus of the present disclosure, there is no intention to preclude melanoidins derived from other food sources from the present disclosure.

Whilst melanoidins are chromophores—molecules in a given composition that absorb particular wavelengths of visible light and in doing so confer color on the composition—certain authors have only made indirect use of that utility in cosmetic compositions.

US 2003068286 A1 (Stroud) describes a self-tanning compositions comprising from 0.01 to 10 wt. % of 1,3-dihydroxyacetone (DHA) and from 0.01 to 10 wt. % of 3,5-dimethoxy-4-hydroxybenzaldehyde. The applied compositions do not comprise melanoidins. Rather this citation proposes that 3,5-dimethoxy-4-hydroxybenzaldehyde produces olive-green colored melanoidins in the upper layers of skin, while the DHA produces orange-brown melanoidins in the upper layers in the skin at approximately the same reaction rate of the 3,5-dimethoxy-4-hydroxybenzaldehyde.

US 2016128919 A1 (Bjork) discloses a device for applying a skin darkening agent, wherein said skin darkening agent is selected from a group consisting of: dihydroxyacetone (DHA), erythrulose, henna, glucose tyrosinate, acetyl tyrosine, brazilin, caffeine, coffee extracts, isobutyl methyl xanthine, methyl xanthine, tea extracts, theophylline, isotin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, or walnut extract, cacao extract, spice extract, fruit extract, vegetable extract, and berry extract. The applied agent does not comprise melanoidins but it is considered that amino acids within skin may react with applied DHA to form melanoidin chromophores.

In contrast to these citations, the present disclosure is concerned with the direct inclusion of melanoidins in cosmetic compositions where said melanoidins can replace—in whole or in part—the organic pigments, mineral pigments and synthetic dyes commonly employed. It would be beneficial, for instance, to replace carbon black about which pigment there is debate about both its deleterious health impacts and its being derived from the incomplete combustion of inter alia non-renewable carboniferous resources. It would furthermore be advantageous to replace azo dyes in cosmetics, of which certain types are allergenic. Moreover, certain azo dyes are decomposed under the action of either ultraviolet radiation or skin microflora: aromatic amine decomposition products may then be deleteriously absorbed by the skin.

As will be further recognized by the skilled artisan, organic synthetic dyes found in cosmetics—that is “FD&C” or “D&C” synthetic dyes—are typically obtained as coal-tar derivatives or from the refining of petroleum oil. These are non-renewable sources. Moreover, even though such organic synthetic dyes may be certified for use by, for example, the United States Food and Drug Administration, this does not preclude the dyes from containing traces of toxic heavy metals. The present inventors have recognized that it would be a significant benefit to derive melanoidin colorants for cosmetic applications from renewable resources and/or waste foodstuffs. In this context it is noted that almost 50% of the worldwide coffee production is processed for soluble coffee preparation, which generates around 6 million tons of spent coffee grounds per year, according to Mussatto et al. Separation and Purification Technology 83: 173-179, 2011. Such spent coffee grounds would represent a highly attractive source material for cosmetic colorants.

Rodriguez et al. “Isolation and Characterization of Melanoidins from Dulce de Leche, A Confectionary Dairy Product” Molecules 2019, 24, 4163 notes that melanoidins can act as antioxidants and can further exhibit genotoxicity, cytotoxicity and antimicrobial activity. For example, the authors here noted that melanoidins may inhibit growth of pathogenic bacteria, such as Helicobacter pylori, Escherichia coli, Bacillus cereus, Staphylococcus aureus, Proteus mirabilis, Pseudomonas aeruginosa and Salmonella typhimurium. The present inventors have recognized that these properties may provide a complimentary activity to the use of melanoidins as chromophores in cosmetic products, wherein melanoidins may replace—in whole or in part—added antioxidants and preservatives, such as formaldehyde, butylated hydroxytoluene (BHT) and butylated hydroxyanisole (BHA).

### STATEMENT OF THE INVENTION

In accordance with a first aspect of the present invention, there is provided a process for obtaining at least one melanoidin fraction from a foodstuff, said process comprising the steps of:
a) providing a particulate foodstuff which comprises melanoidins;
b) treating said particulate foodstuff with an aqueous extractant solution having a pH greater than 7 to extract melanoidins as solute in the aqueous phase;
c) physically separating said aqueous phase from the residual treated particulate foodstuff;
d) fractionating the dispersed or dissolved components of said aqueous phase on the basis of molecular weight; and,
e) dehydrating said fractionated aqueous phase to obtain at least one solid melanoidin fraction therefrom.

The particulate foodstuff is preferably selected from spent coffee grounds and Brewers Spent Grain (BSG). The particulate foodstuff may, independently or additionally to this condition, be micronized.

In an embodiment of the invention, said fractionation step d) is performed by: ultra-filtration using molecular weight cut-off membranes; centrifugation; or, size exclusion chromatography. In a further, but not mutually exclusive embodiment, said dehydration step e) is performed by at least one of: freeze-drying; spray drying; convective drying; radiation drying; and, vacuum drying.

In accordance with a second aspect of the present invention, there is provided a cosmetic composition comprising melanoidins, wherein said melanoidins are characterized by having a weight average molecular weight (Mw) of from 200 daltons (Da) to 300 kilodaltons (kDa). The melanoidins may be characterized by a weight average molecular weight (Mw) of from 1 to 300 kDa, preferably from 1 to 100 kDa and more preferably from 10 to 100 kDa. For example, said melanoidins should be characterized by having a weight average molecular weight (Mw) of either from 10 to 30 kDa, from 30 to 50 kDa or from 50 to 100 kDa. It is preferred that said melanoidins included in the cosmetic compositions are obtained by the process defined hereinabove and in the appended claims. The included melanoidins may be present in the compositions in un-encapsulated form.

The cosmetic composition as defined herein above and in the appended claims may be a non-powdered solid cosmetic composition, a compact powder cosmetic composition or an aqueous liquid cosmetic composition. Illustrative but important embodiments of the cosmetic composition are constituted by the provision of: an aqueous hair dyeing composition; an aqueous tattoo ink composition; and, a solid composition which may have utility as facial and, in particular, eye-make-up.

In a particularly preferred embodiment, the aforementioned cosmetic compositions are characterized as being substantially free of caffeine.

### Definitions

As used herein, the singular forms “a”, “an” and “the” include plural referents unless the context clearly dictates otherwise.

The terms “comprising”, “comprises” and “comprised of” as used herein are synonymous with “including”, “includes”, “containing” or “contains”, and are inclusive or open-ended and do not exclude additional, non-recited members, elements or method steps. If used, the phrase “consisting of” is closed, and excludes all additional elements. Further, the phrase “consisting essentially of” excludes additional material elements, but allows the inclusions of non-material elements that do not substantially change the nature of the invention.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

Further, in accordance with standard understanding, a weight range represented as being “from 0 to x” specifically includes 0 wt. %: the ingredient defined by said range may be absent from the composition or may be present in the composition in an amount up to x wt. %.

The words “preferable”, “preferred”, “preferably”, “particularly” and “desirably” are used frequently herein to refer to embodiments of the disclosure that may afford particular benefits, under certain circumstances. However, the recitation of one or more preferable, preferred, particular or desirable embodiments does not imply that other embodiments are not useful and is not intended to exclude those other embodiments from the scope of the disclosure.

As used throughout this application, the word “may” is used in a permissive sense—that is meaning to have the potential to—rather than in the mandatory sense.

As used herein, room temperature is 23° C. plus or minus 2° C.

As used herein, number average molecular weight (Mn) and weight average molecular weight (Mw) are determined by gel permeation chromatography (GPC) with tetrahydrofuran (THF) as the eluent in accordance with DIN 55672-1:2007-08.

The term “micronized” when used in reference to a foodstuff, means a powder having a particle size of from 1 to 1500 microns, typically from 100 to 1250 microns. The term “micronized” is intended to refer both to: particles which have been produced through finely dividing materials which are originally presented in bulk form; and, particles obtained by other mechanical, chemical or physical methods, including formation in solution with or without a seeding and comminution by one or more of pulverization, milling, grinding, homogenization and sonication.

Grinding can, for instance, be effected using a planetary ball mill having a grinding chamber that includes a rotor shaft that is used to rotate grinding media. Reference may be made to: Le Caer et al. Mechanical Alloying and High-Energy Ball-Milling: Technical Simplicity and Physical Complexity for the Synthesis of New Materials www.ademe.fr/recherche/manifestations/materiaux_2002/.

Milling may be performed in any high-energy mill, of which examples include: centrifugal mills; planetary ball mills; jet mills, such as spinning air flow jet mill; and, fluid energy mills. Desirably, the high-energy mill should be able to impart an impact force of at least 0.5 G, for example from 0.5 to 25 G, to the milling media. Non-limiting examples of mills which may find utility in the present invention are disclosed in: U.S. Pat. Nos. 5,522,558; 5,232,169; 6,126,097; and, 6,145,765. Moreover, it should be noted that the present invention does not preclude milling being conducted under heat—such as described in WO00/56486—and/or in the presence of additives, such as lubricants, surfactants, dispersants and solvents.

A typical sonication would first comprise adding said particles to at least one solvent and optionally at least one reactant. The employed solvent(s) should comprise or consist of a non-polar solvent selected from the group consisting of: alkanes (R—H); cyclic alkanes; branched alkanes; aromatics (Ar—H); alkyl halides (R—X); and, mixtures thereof. Exemplary but non-limiting non-polar solvents include n-pentane, n-hexane, cyclohexane, n-heptane, isooctane, trimethylpentane, toluene, xylene and benzene. The presence of reactants in addition to said solvents is preferred: reactants—such as silylating agents and organofunctional silanes—serve to pacify the newly generated particle surfaces created during sonication and the fragmentation of the starting particles and to thereby yield unreactive particles.

Sonic energy is then applied to the formed medium. As will be recognized by the skilled practitioner, the frequency of sonication, the time of sonication and the power used are key determinants for the end particle size distribution. For illustration only, MisoNix Sonicator® 3000 available from Cole-Parmer Instrument Company may be mentioned as an exemplary sonic probe for performing sonication.

After comminuting, any fluid present in the comminution step(s) may be separated from the particles. One or more separation process, such as air-drying, heating, filtration and evaporation, can be employed in this regard but it is preferred that the fluid is removed under heating to a sufficient temperature to prevent agglomeration of the particle during the drying thereof.

As used herein, by “d₅₀ particle size” is meant that the particle size distribution is such that at least 50% of the particles by weight have a particle size diameter of less than the specified value. Unless otherwise stated, that particle size is determined by dynamic light scattering.

Viscosities of the compositions described herein are, unless otherwise stipulated, measured in accordance with ASTM D3236 using the Brookfield Viscometer at standard conditions of 25° C. and 50% Relative Humidity (RH). The method of calibration, the spindle type and rotation speed of the Brookfield Viscometer are chosen according to the instructions of the manufacturer as appropriate for the composition to be measured.

The term “cosmetic” is used herein in accordance with the definition applied in US Federal Food, Drug and Cosmetic Act—as Amended Through P. L. 107-377, 19 Dec. 2002—as “articles intended to be rubbed, poured, sprinkled, or sprayed on, introduced into, or otherwise applied to the human body . . . for cleansing, beautifying, promoting attractiveness, or altering the appearance”. In its broadest disclosure, the form of the cosmetic composition of the present invention is not particularly limited and may be a liquid, emulsion, paste, gel, solid or compact powder. Particular examples of the cosmetic composition include: facial cleansers; facial treatments such as skin lotion, skin milk, cream, gel, serum and facial masks; facial and eye make-up preparations, such as face powder, foundation, lip rouge, blush, eyeliner, mascara, eye shadow and eyebrow pencil; tattoos, including permanent makeup; hair cosmetic compositions such as shampoo, rinse, conditioner, hair styling agent, hair treatment and hair colorants. Included in particular within this definition are: eye and facial makeup preparations; tattoos, including permanent makeup; and, hair colors.

The term “solid” as used herein with respect to cosmetic compositions references the state of the composition at room temperature and at atmospheric pressure: the composition possesses a high consistency which conserves its form during storage and does not flow under its own weight.

The term “structurant”, as used herein, means any material known or otherwise effective in providing suspending, gelling, viscosifying, solidifying and/or thickening properties to the composition, or those materials which otherwise provide structure to the final product form. These solid structurants include one or more solid crystalline or other nonpolymeric suspending agents suitable for topical application to human skin. Suitable suspending agents are those that can form in the composition a crystalline or other matrix within which volatile solvents, non-volatile solvents, or other liquid components of the composition are contained. Such materials will typically be solids under ambient conditions and include organic solids, waxes, crystalline or other gallants, or combinations thereof. A structurant provides a uniform distribution of the particulate active throughout the product and also controls product hardness or rheology. For completeness, crystalline structurants suitable for use in the solid compositions disclosed herein are described inter alia in U.S. Pat. No. 5,552,136, (Motley), U.S. Pat. No. 5,976,514 (Guskey et al.), and U.S. Pat. No. 5,891,424 (Bretzler et al.).

The term “compact powder” refers to a mass of product whose cohesion is at least partly provided by compacting or pressing during manufacture. In particular, by taking a measurement using a TA.XT.plus Texture Analyser texturometer—available from Stable Micro Systems—the compact powder should have a pressure resistance of between 0.1 and 2.5 kg relative to the surface area of the spindle used. The measurement of this resistance is performed by moving an SMS P/3 flat-headed cylindrical spindle (7.07 mm²) over a distance of 1.5 mm and at a speed of 0.5 mm/sec.

The term “tattoo” references an indelible mark or figure on the mammalian body obtained through the insertion a pigment under the skin using needles, scalpels, or other related equipment. The term is intended to encompass both intra-epidermal tattoos and intra-dermal tattoos which will be distinguished inter alia by their degree of permanence.

As used herein, “keratinous tissue” refers to the keratin-containing layers disposed as the outermost protective covering of mammals which include, but are not limited to, skin, hair, nails, and cuticles. There is no particular intention to limit the locus of the keratinous tissue to which the present cosmetic compositions may be applied: mention may be made of keratinous tissue disclosed on the face, neck, chest, back, arms (including underarms), hands, legs, feet, buttocks and scalp.

The term “epidermis” as used herein refers to the outer layer of skin of which the predominant cell type is the keratinocyte. As would be understood by the skilled artisan, the epidemis is conventionally divided into five strata: stratum corneum; stratum granulosum; stratum spinosum; and stratum basale. The stratum corneum is often referenced as being non-viable and contains many layers of dead, anucleated keratinocytes that are essentially filled with keratin. The stratum lucidum contains two to three layers of anucleated cells. The stratum granulosum contains two to four layers of cells that are held together by desmosomes that contain keratohyaline granules. The stratum spinosum contains eight to ten layers of modestly active dividing cells that are also held together by desmosomes. The stratum basale contains a single layer of columnar cells that actively divide by mitosis and provide the cells that are destined to migrate through the upper epidermal layers to the stratum corneum.

The term “dermis” as used herein refers to the layer of skin between the epidermis and the subcutaneous tissues. The dermis provides structural support to the skin and comprises non-cellular collagen fibers derived from fibroblasts.

The term “hair” herein means mammalian keratin fibers and includes scalp hair, facial hair and body hair. In addition, the term encompasses hair both hair which is attached to a living subject and also hair that has been removed there from. For completeness, a “hair shaft” or “hair fiber” means an individual hair strand.

The expression “proximal to the scalp” means that portion of an extended, or substantially straightened, hair shaft that is closer in distance to the scalp than to the end of the hair. Thereby 50% of the hair fiber length would be considered proximal to the scalp and 50% of the hair fiber length would be distal to the scalp. When used “x cm” proximal to the scalp means a distance “x” along an extended or substantially straightened hair measured from the scalp as the endpoint.

“Cosmetically acceptable” means that the compositions, formulations or components described herein are suitable for use in contact with human keratinous tissue without undue toxicity, incompatibility, instability, allergic response, and the like. All compositions and formulations described herein which have the purpose of being directly applied to keratinous tissue are limited to those being cosmetically acceptable.

The term “topical application”, as used herein, means to apply or spread the compositions of the present invention onto the surface of the skin.

Water, for use as a (co-)solvent herein, is intended to mean water of low solids content as would be understood by a person of ordinary skill in the art. The water may, for instance, be distilled water, demineralized water, deionized water, reverse osmosis water, boiler condensate water, or ultra-filtration water. Tap water may be tolerated in certain circumstances but is not tolerated within the tattoo ink composition described herein.

As used herein “solvents” are substances capable of dissolving another substance to form a uniform solution; during dissolution neither the solvent nor the dissolved substance undergoes a chemical change. Solvents may either be polar or non-polar. The term “alcoholic solvent” encompasses such solvents which are any water-soluble mono-alcohols, diols or polyols that are liquids at 25° C. at atmospheric pressure.

If an organic liquid product has a boiling point region, then the onset (the lowest temperature) of the boiling point range at atmospheric pressure is taken as the nominal boiling point. Where necessary any measurement of the initial boiling point for materials should be conducted in accordance with ASTM Standard Test Method D1078-95, or its most current version.

The term “water-miscible organic solvent”, as used herein, refers to an organic solvent that is completely miscible with water at room temperature. In this regard, a particular preference may be noted for organic solvents which are soluble, freely soluble or very soluble in water and are thereby characterized by requiring ≤30 ml of water to dissolve 1 g of organic solvent at room temperature (https://wmw.sigmaaldrich.com/united-kingdom/technical-services/solubility.html).

The term “water-immiscible organic solvent”, as used herein refers to organic solvents that form a two-phase system with water. In this regard, a particular preference may be noted for organic solvents which are slightly soluble, very slightly soluble or practically insoluble in water and are thereby characterized by requiring ≥100 ml of water to dissolve 1 g of organic solvent at room temperature (https://www.sigmaaldrich.com/united-kingdom/technical-services/solubility.html).

As used herein, the term “dispersion” refers to a composition that contains discrete particles that are distributed throughout a continuous liquid medium.

As used herein, the term “surface tension” refers to the force required to increase the unit area of a surface of a liquid or of an interface between two liquids or between a liquid and a gas, generally stated in units of dynes/cm. Surface tensions described herein are measured by the Du Noüy ring method utilizing an EasyDyne tensiometer model K20 marketed by Krüss USA, Matthews, N.C.

As used herein, “C₁-Cₙ alkyl” group refers to a monovalent group that contains 1 to n carbons atoms, that is a radical of an alkane and includes straight-chain and branched organic groups. As such, a “C₁-C₆ alkyl” group refers to a monovalent group that contains from 1 to 6 carbons atoms, that is a radical of an alkane and includes straight-chain and branched organic groups. Examples of alkyl groups include, but are not limited to: methyl; ethyl; propyl; isopropyl; n-butyl; isobutyl; sec-butyl; and, tert-butyl. In the present invention, such alkyl groups may be unsubstituted or may be substituted with one or more halogen. Where applicable, a preference for a given substituent will be noted in the specification.

The term “C₂-C₆ alkylene” as used herein, is defined as saturated, divalent hydrocarbon radical having from 2 to 6 carbon atoms.

The present compositions are defined herein as being “substantially free” of certain compounds, elements, ions or other like components. The term “substantially free” is intended to mean that the compound, element, ion or other like component is not deliberately added to the composition and is present, at most, in only trace amounts which will have no (adverse) affect on the desired properties of the composition. An exemplary trace amount is less than 1000 ppm by weight of the composition. The term “substantially free” encompasses those embodiments where the specified compound, element, ion, or other like component is completely absent from the composition or is not present in any amount measurable by techniques generally used in the art.

### BRIEF DESCRIPTION OF THE FIGURES

The disclosure will make reference to the appended drawing in which:

FIG. 1 illustrates an embodiment of the preparation of a cosmetic composition comprising a melanoidin fraction.

### DETAILED DESCRIPTION OF THE INVENTION

The present invention provides for the use in cosmetic compositions of a melanoidin fraction characterized by its molecular weight. Most broadly, said melanoidins are characterized by having a weight average molecular weight (Mw) of from 200 daltons (Da) to 300 kilodaltons (kDa). The melanoidins may, for instance, have a weight average molecular weight of from 1 to 300 kDa, for example from 1 to 200 kDa, from 1 to 100 kDa or desirably from 10 to 100 kDa. It may be practical to employ melanoidin fractions which possess a more narrowly defined molecular weight, such as from 10 to 30 kDa, from 30 to 50 kDa, from 50 to 100 kDa or from 100 to 200 kDa where, for example, a particular pigmentary or light absorption effect is desired. It is also envisaged that more than one melanoidin fraction may be present: for example, a first fraction based on melanoidins possessing a weight average molecular weight of from 10 to 30 kDa may be combined in the cosmetic composition with a second fraction based on melanoidins having a weight average molecular weight of from 50 to 100 kDa or from 100 to 200 kDa.

Whilst the present disclosure does not preclude other means of obtaining said melanoidin fractions, the present invention does provide a process for obtaining at least one melanoidin fraction from a foodstuff, said process comprising the steps of:
a) providing a particulate foodstuff which comprises melanoidins;
b) treating said particulate foodstuff with an aqueous extractant solution having a pH greater than 7 to extract melanoidins as solute in the aqueous phase;
c) physically separating said aqueous phase from the residual treated particulate foodstuff;
d) fractionating the dispersed or dissolved components of said aqueous phase on the basis of molecular weight; and,
e) dehydrating said fractionated aqueous phase to obtain at least one solid melanoidin fraction therefrom.

Exemplary particulate foodstuff sources which may be provided to step a) include coffee, cocoa, bread, malt, barley, Brewers Spent Grain (BSG), soy, meat and honey. Said coffee may itself be constituted by roasted coffee beans, coffee grounds, spent coffee grounds (SCG), coffee silverskin (CS) and granulated coffee extract. Spent Brewers Grains (SBG) are the solid residues left after the processing of germinated and dried cereal grains for the production of beer and other malt products, such as malt extracts and malt vinegar: whilst barley is the main grain used for brewing, beers are also made from wheat, maize, rice, sorghum and millet. Spent coffee grounds (SCG) are the residual material obtained during the treatment of coffee grounds with hot water or steam for the instant coffee preparation. Coffee silverskin (CS) is a thin tegument of the outer layer of green coffee beans obtained as a by-product of the roasting process.

In the present disclosure, it is preferred that the particulate foodstuff is micronized. Independently or additionally, it is preferred that the particulate foodstuff is selected from spent coffee grounds and Brewers Spent Grain (BSG).

A particular preference for micronized spent coffee grounds might be noted. As regards this embodiment, the present method may encompass at least one pre-treatment step which precedes step b). It may be advantageous, for example, to dry the spent coffee grounds by exposure to a temperature condition above room temperature, such as from 50 to 100° C. Alternatively or additionally to this drying stage, the spent coffee grounds may be defatted. At least a portion of the lipids and/or oils present in the grounds may be extracted using a suitable solvent: mention may be made of carbon dioxide applied at a pressure and temperature condition such that it is a supercritical fluid.

Treatment step b) may be performed using any method which provides for the performance of the following stages: (1) the penetration of the aqueous extractant into the solid matrix of the provided or pre-treated particles; (2) the dissolution of the solute in the aqueous extractant; (3) the diffusion of the solute out from the solid matrix; and, (4) the collection of the extracted solutes. Suitable methods include but are not limited to: centrifugation; maceration; percolation; decoction; reflux extraction; pressurized liquid extraction; ultrasound assisted extraction; microwave assisted extraction; pulsed electric field extraction; and, hydro-distillation. For example, a suitable centrifugation method may comprise the addition of the aqueous extractant to the particulate foodstuff followed by the application of from 500 to 5000 gravity (G), for a period of from 5 to 60 minutes at a temperature of from 5 to 30° C.: the supernatants containing the melanoidins may then be collected. In a further example, the particulate foodstuff may be treated by pressurized liquid extraction at a temperature of from 100° to 200° C. for a period of from 10 to 120 minutes, for example from 20 to 60 minutes.

Factors which enhance the diffusivity and solubility of the melanoidins will facilitate their extraction. Herein, it is preferred that the aqueous treatment solution has a pH of above 7 but less than or equal to 12 or less than or equal to 10. The aqueous treatment solution may, alternatively or additionally to those preferred pH ranges, be an alkaline solution comprising at least one of: sodium hydroxide; potassium hydroxide; ammonia (NH_{3(aq)}); and, calcium hydroxide.

The presence of a water-miscible solvent within the aqueous extractant solution is not precluded. Such a water-miscible solvent should not however constitute more than 20 wt. % of the aqueous extractant solution.

After separation of the aqueous phase from the aqueous phase from the residual treated particulate foodstuff (step c)) —by, for example, filtration, precipitation or decantation—the dispersed or dissolved components are fractionated on the basis of their molecular weight. This fractionation, which optionally may be preceded by the concentration of the aqueous solution or dispersion, may be effected by ultra-filtration using requisite molecular weight cut-off membranes, which technique yields a plurality of retentates which may then be further processed. Fractionation may also be effected by centrifugation or size exclusion chromatography. It will be recognized that the appropriate selection of the minimum molecular weight of the compounds to be fractionated can substantially eliminate caffeine (Mw 194 da) from the obtained retentates.

The fractionated products, such as the aforementioned retentates, are then subjected to a water removal step to yield the melanoidin fractions in solid form. Suitable methods of dehydration which may be mentioned include: freeze-drying; spray-drying; convective drying; radiation drying; and, vacuum drying. The obtained solid melanoidin fractions may be presented as particles having an average particle size (d50) of from 1 to 500 μm. Where necessary particle aggregates may be comminuted to attain this particle size.

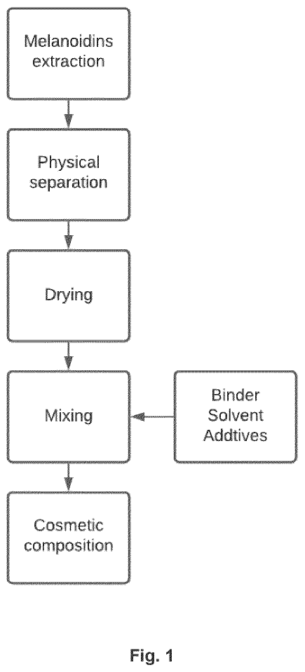

As depicted in FIG. 1 appended hereto, the separated and dried melanoidins, fractionated on the basis of molecular weight, are combined with further ingredients to attain a cosmetic composition. The further ingredients have been identified as being a binder, solvent and additives in the appended FIGURE: this is not intended to be limiting as the exact nature of the added ingredients will dependent on the type and form of the cosmetic composition. Any added ingredients should result in the formation of a cosmetically acceptable composition.

The compositions of the present disclosure are formulated by simple mixing of the various components—as well as any adjunct ingredients—in pre-determined amounts. This may be performed using any of the mixing techniques known in the art. It would certainly be preferred however that the ingredients are not mixed by hand but are instead mixed by machine—a static or dynamic mixer, for example—in order to ensure a homogenous dispersion of: particulate ingredients; oleaginous ingredients, where said cosmetic composition is an oil-in-water emulsion; and, water and water-borne ingredients, where said cosmetic composition is a water-in-oil emulsion.

If necessary, the compositions may be prepared well in advance of their application. However, in an interesting alternative embodiment for aqueous cosmetic compositions, a concentrated composition may first be obtained by mixing components with only a fraction of the water that would be present in the composition as applied: the concentrated composition may then be diluted with the remaining water shortly before its application. It is considered that such concentrated compositions may be prepared and stored as either single-package concentrates—that can be converted by dilution with water only—or as multi-part concentrates, two or more of which must be combined and diluted to form a complete working composition according to the invention. Any dilution can be effected simply by the addition of water, in particular deionized and/or demineralized water, under mixing. The composition might equally be prepared within a rinse stream whereby one or more streams of the concentrate(s) is injected into a continuous stream of water.

Various features of the disclosure are described in the following exemplary embodiments, which are intended to be representative and not limiting.

### Exemplary Embodiment 1: Aqueous Hair Dyeing Composition

In an important embodiment of the present disclosure there is provided an aqueous hair dyeing composition, comprising:
a) melanoidins, wherein said melanoidins have a weight average molecular weight of from 200 daltons to 300 kDa, preferably from 1 to 300 kDa and more preferably from 1 to 100 kDa, for example from 10 to 100 kDa; and,
b) at least one auxiliary agent selected from the group consisting of: wetting agents; swelling agents; penetrants; pH regulators; surfactants; thickeners; antimicrobial agents; and, perfumes.

Component a) is preferably obtained from the extractive process as defined herein above and in the appended claims. For example, the hair dyeing composition of the present invention may comprise, based on the weight of the composition:
from 5 to 20 wt. % of a) melanoidins, said melanoidins having a weight average molecular weight of from 200 daltons to 300 kDa, preferably from 1 to 300 kDa and more preferably from 1 to 100 kDa, for example from 10 to 100 kDa;
from 5 to 40 wt. % of b) at least one auxiliary agent selected from the group consisting of: wetting agents; swelling agents; penetrants; pH regulators; surfactants; thickeners; antimicrobial agents; and, perfumes; and,
from 40 to 90 wt. % of water.

It will be noted that component b) presents categorizations based on the benefit provided by the agent or the agent's postulated mode of action. This categorization is made for convenience: a given chemical ingredient—such as ammonia for example—might provide more than one benefit or operate via more than one mode of action.

As regards the aqueous hair dye compositions, it is preferred that said compositions contain from 40 to 90 wt. %, preferably from 40 to 80 wt. % and more preferably from 55 to 75 wt. %, based on the weight of the composition, of water. In an alternative but not mutually exclusive characterization, the aqueous hair dye composition may be defined by a viscosity of from 0.005 to 50 Pa·s, as measured using a Brookfield viscometer at 25° C.

Exemplary wetting agents, which may be used alone or in combination, include but are not limited to: glycerin; propylene glycol; sorbitol; 1,3-butylene glycol; polyvinylpyrollidone (PVP); polyethylene glycols (PEG); polypropylene glycol (PPG); PEG/PPG-block copolymers; and, PEG/PPG-random copolymers. An illustrative PEG/PPG-random copolymer is PEG/PPG—8/17.

Exemplary swelling agents include ammonia and monoethanolamine (MEA). The ammonia, when included, would be present in the aqueous compositions of the present invention as an ammonia solution NH₃(aq) which encompasses weakly basic solutions of ammonia in water which may referred to in the art as ammonium hydroxide, ammonia water, ammonia liquor, aqua ammonia, aqueous ammonia, or simply ammonia. While the term “ammonium hydroxide” suggests a base with the composition [NH₄⁺][OH⁻], it is virtually impossible to isolate samples of NH₄OH, insomuch as these ions do not comprise a significant fraction of the total amount of ammonia in an ammonia solution, except in the case of extremely dilute ammonia solutions.

Exemplary penetrants, which may be used alone or in combination include: monohydric alcohols having C₁-C₆ alkyl group such as ethanol, 1-propanol, 2-propanol, 1-butanol, and 2-butanol; polyhydric alcohols having from 3 to 8 carbon atoms such as propanediol, butanediol, pentanediol, hexanediol, hexanetriol, heptanediol, heptanetriol, octanediol, octanetriol, isoprene glycol, propylene glycol, glycerin, and diethylene glycol monoethyl ether; esters of said polyhydric alcohols; N-alkypyrrolidones which are liquid at room temperature such as N-methyl-2-pyrrolidone, N-ethyl-2-pyrrolidone, N-propyl-2-pyrrolidone, N-butyl-2-pyrrolidone, and N-cyclohexyl-2-pyrrolidone; C₂-C₆ alkylene carbonates such as ethylene carbonate and propylene carbonate; aromatic alcohols such as benzyl alcohol, benzyloxyethanol, cinnamyl alcohol, p-anisyl alcohol, p-methylbenzyl alcohol, phenoxyethanol, phenoxyisopropanol, 2-benzyl ethanol and β-phenylethyl alcohol. A preference for 2-propanol, 1,2-hexanediol and benzyl alcohol might be mentioned.

Examples of pH adjusters include: mineral acids, such as phosphoric acid; hydroxycarboxylic acids such as lactic acid, glycolic acid, citric acid, 3-hydroxybutyric acid, 4-hydroxybutyric acid, 2-hydroxybutanedioic acid, 2,3-dihydroxybutanedioic acid, 3-hydroxyvaleric acid, 5-hydroxyvaleric acid, 6-hydroxycaproic acid and the alkali metal or alkaline earth metal salts thereof; ammonia; alkali metal hydroxides; alkali metal carbonates; alkali metal bicarbonates; alkaline earth metal hydroxides; alkaline earth metal carbonates; and, alkaline earth metal bicarbonates.

The aqueous hair dye compositions of the present invention will typically comprise up to 10 wt. %, for example up to 5 wt. % or up to 3 wt. %, based on the weight of the composition, of surfactant. A wide variety of surfactants may be used herein—both for emulsification of the dispersed phase and to provide acceptable spreading of the applied composition—but desirable surfactants will be selected from the group consisting of: anionic surfactants; nonionic surfactants; amphoteric surfactants; non-lathering surfactants; emulsifiers; and, mixtures thereof. U.S. Pat. No. 6,280,757 (McAtee et al.) provides an instructive disclosure on suitable surfactants.

Anionic Surfactants: Non-limiting examples of anionic surfactants useful in the compositions of the present invention are disclosed U.S. Pat. No. 3,929,678 (Laughlin et al.) and U.S. Pat. No. 4,557,853. Mention may be made of anionic surfactants selected from the group consisting of: i) fatty acid soaps based on fatty acids having from 8 to 36 or from 8 to 24 carbon atoms; ii) monoalkyl, dialkyl, and trialkylphosphate salts; iii) sarcosinates, such as sodium lauroyl sarcosinate, sodium myristoyl sarcosinate and sodium cocoyl sarcosinate; iv) sulfates, including alkyl and alkyl ether sulfates of which mention may be made of sodium lauryl sulfate, ammonium lauryl sulfate, ammonium laureth sulfate, sodium laureth sulfate, sodium trideceth sulfate, ammonium cetyl sulfate and sodium cetyl sulfate; v) isethionates such as sodium lauroyl isethionate and ammonium cocoyl isethionate; vi) taurates such as sodium lauroyl methyl taurate and sodium cocoyl methyl taurate; vii) lactylates such as sodium lauroyl lactylate, triethanolamine lauroyl lactylate and sodium caproyl lactylate; viii) glutamates such as sodium lauroyl glutamate, sodium myristoyl glutamate, and sodium cocoyl glutamate; and, mixtures thereof.

Non-Ionic Surfactants: Suitable nonionic surfactants having utility in the present disclosure include: alkyl glucosides; alkyl polyglucosides; polyhydroxy fatty acid amides; alkoxylated fatty acid esters; sucrose esters; amine oxides; and. mixtures thereof. Mention may, for example, be made of: C₈-C₁₄ glucose amides, C₈-C₁₄ alkyl polyglucosides, sucrose cocoate, sucrose laurate, lauramine oxide, cocoamine oxide and mixtures thereof.

Amphoteric Surfactants: The term “amphoteric surfactant” as used herein, is also intended to encompass zwitterionic surfactants. Useful amphoteric surfactants, which may be used alone or in combination, include but are not limited to: derivatives of aliphatic secondary and tertiary amines, preferably wherein the nitrogen is in a cationic state and wherein at least one aliphatic radical contains an ionizable water solubilizing group such as a carboxyl, sulfonate, sulfate, phosphate or phosphonate group; betaines; sultaines; hydroxysultaines; alkyliminoacetates; iminodialkanoates; and, aminoalkanoates.

Non-Lathering Surfactants: Given that the present hair dye compositions are not conventionally intended to have a cleansing functionality, they need not therefore be lathering systems and can comprise non-lathering surfactants. Exemplary non-lathering surfactants having utility herein include: polyethylene glycol 20 sorbitan monolaurate (Polysorbate 20); Polysorbate 60; Polysorbate 80; polyoxyethylene 20 sorbitan trioleate (Polysorbate 85); Steareth-20; Ceteth-10; Ceteareth-20; cetyl phosphate; potassium cetyl phosphate; diethanolamine cetyl phosphate; glyceryl stearate; PPG-2-methyl glucose ether distearate; and, PEG-100 stearate.

Emulsifier systems: In addition to the above, the use of emulsifier(s) is not precluded in the present disclosure. Mention may be made of the following emulsifier mixtures: PROLIPID® 141 (glyceryl stearate, behenyl alcohol, palmitic acid, stearic acid, lecithin, lauryl alcohol, myristyl alcohol and cetyl alcohol) available from ISP; PROLIPID® 151 (Glyceryl stearate, cetearyl alcohol, stearic acid, 1-propanamium, 3-amino-N-(2-(hydroxyethyl)-N—N-Dimethyl,N—C(16-18) Acyl Derivatives, Chlorides), available from ISP; POLAWAX® NF (Emulsifying wax NF) available from Croda; INCROQUAT® BEHENYL TMS (behentrimonium sulfate and cetearyl alcohol) available from Croda; and EMULLIUM® DELTA (cetyl alcohol, glyceryl stearate, peg-75 stearate, ceteth-20 and steareth-20) available from Gattefosse.

The aqueous hair dye compositions of the present disclosure may comprise from 0 to 10 wt. %, for example from 0 to 5 wt. %, based on the weight of the composition, of one or more thickening agents. Said thickening agents may be selected from: i) carboxylic acid polymers, such as those members of the CARBOPOL®900 series available from B.F. Goodrich; ii) acrylates/C₁₀-C₃₀ alkyl acrylate crosspolymers, such as CARBOPOL®1342, CARBOPOL®1382, PEMULEN® TR-1 and PEMULEN® TR-2 available from B.F. Goodrich; iii) crosslinked polyacrylate polymers, which may be cationic or nonionic polymer; iv) polyacrylamide polymers, in particular nonionic polyacrylamide polymers; iv) multi-block copolymers of acrylamides and substituted acrylamides with acrylic acids and substituted acrylic acids; v) polysaccharides including scleroglucans, cellulose and cellulose derivatives, such as carboxymethylcellulose and alkyl hydroxyalkyl cellulose ethers; vi) modified starches; vii) gums such as xantham gum, acacia gum, alginin, sodium alginate, locust bean gum and guar gum; viii) proteins such as collagen, albumen and gelatin; and, mixtures thereof.

The aqueous hair dye compositions of the present disclosure may comprise from 0 to 5 wt. %, preferably from 0 to 2 wt. %, based on the weight of the composition, of at least one antimicrobial agent. It is preferred that the or each antimicrobial agent included in the composition is a crystalline particulate that is insoluble in water. Exemplary antimicrobial agents include but are not limited to: sulfur; piroctone olamine; selenium sulfides, as described in U.S. Pat. Nos. 2,694,668, 3,152,046 and 4,089,945; and, pyridinethione salts, as described in U.S. Pat. Nos. 3,753,196, 4,345,080, 4,323,683, and 4,470,982.

Examples of perfumes which may be included in the aqueous hair dyeing composition include vanillin, cinnamic alcohol, heliotropine, coumalin, 2-methyl-3-(3,4-methylenedioxy-phenyl)-propanal, 4-(4-hydroxyphenl)-2-butanone, benzaldehyde, anisyl alcohol, 3,4-di methoxybenzaldehyde, heliotropyl acetate, phenyl acetaldehyde dimethylacetal, phenoxyethyl alcohol, phenyl acetaldehyde glycerylacetal, benzyl alcohol, phenylethyl alcohol, furaneol, sugar lactone, menthol, ethyl diglycol, benzyl acetate, linalool, camphor, terpineol, citronellol, geraniol, 2,6-nonadienol, methyloctyl carbonate, 3,7-dimethyl-2,6-octadienal and nonanal.

The hair dye of the present composition may be applied using conventional means to at least a portion of the hair shaft, including by finger touch, by hand, by brush or by another implement, optionally in concert with a means of delivering thermal energy or a suitable electromagnetic radiation. The composition(s) may be applied to wet hair or to dry hair. The amount applied will vary, dependent upon the thickness and length of the hair, and the desired effect.

The hair dye composition(s) may be applied to substantially all of the hair or alternatively to a portion of the hair. In one embodiment, the composition may be applied to a portion of the hair that is proximal to the scalp, for example, from 0 to 10 cm or from 0 to 5 cm proximal to the scalp. This may be desirable for the user modify the color of the roots of newly grown hair.

### Exemplary Embodiment 2: Tattoo Ink

In an important embodiment of the present disclosure there is provided an aqueous tattoo ink composition composition, comprising:
a) at least one colorant, wherein said at least one colorant comprises melanoidins, said melanoidins having a weight average molecular weight of from 200 daltons to 300 kDa, preferably from 1 to 300 kDa and more preferably from 1 to 100 kDa, for example from 10 to 100 kDa;
optionally b) at least one co-solvent; and,
c) at least one auxiliary selected from the group consisting of: binders; thickeners; pH regulators; surfactants; humectants; antimicrobial agents; anti-inflammatory agents; anti-oxidants; antiseptics; and, local anesthetics.

The melanoidins present in the colorant component a) are preferably obtained from the extractive process as defined herein above and in the appended claims. It is noted that the colorant component a) may comprise, consist essentially of or consist of said melanoidins depending on the color effect to be obtained.

In a preferred embodiment, the aqueous tattoo ink composition of the present invention comprises, based on the weight of the composition:
from 10 to 40 wt. % of water;
from 30 to 60 wt. % of a) said at least one colorant, wherein said at least one colorant comprises melanoidins, said melanoidins having a weight average molecular weight of from 200 daltons to 300 kDa, preferably from 1 to 300 kDa and more preferably from 1 to 100 kDa, for example from 10 to 100 kDa;
from 0 to 15 wt. % of b) said co-solvent; and,
from 5 to 15 wt. % of c) at least one auxiliary selected from the group consisting of: binders; thickeners; pH regulators; surfactants; humectants; antimicrobial agents; anti-inflammatory agents; anti-oxidants; antiseptics; and, local anesthetics.

It will be noted that component c) presents categorizations based on the benefit provided by the agent or the agent's postulated mode of action. This categorization is made for convenience: a given chemical ingredient—such as polyethylene glycol for example—might provide more than one benefit or operate via more than one mode of action. In this regard, it is repeated here that melanoidins themselves provide a pigmentary function and act as antimicrobial agents, anti-inflammatory agents and anti-oxidants and can thereby minimize the need to provide adjunct ingredients having these functionalities.

The aqueous tattoo ink composition of the present invention can include further colorants in addition to the aforementioned melanoidins. Said colorants are by necessity water-soluble or water-dispersible and may be selected from: inorganic pigments; organic pigments; natural dyes; synthetic dyes; and, combinations thereof. The use of organic, lacquered or lacquer pigments (“lake pigments”) —which are obtained by precipitating a natural or synthetic dye with a metal salt—is also envisaged. It is however preferred that the formulation is substantially free of metal particles and/or is substantially free of microencapsulated colorants.

For completeness, exemplary inorganic pigments include but are not limited to: metal oxides, such as iron oxide red, iron oxide yellow, iron oxide black, anatase, brookite, rutile, aluminum oxide, zirconium oxides, cobalt oxides, cerium oxides, nickel oxide, chromium oxide, nickel-chromium oxides, zinc oxides and composite oxides; metal hydroxides, such as calcium hydroxide, iron hydroxides, aluminum hydroxide, chromium hydroxide, magnesium hydroxide and composite metal hydroxides; Prussian blue; iron sulfide; manganese violet; carbon black; mica; and, kaolin.

The aqueous tattoo ink composition may comprise at least one water-miscible organic co-solvent. When present, the water and said at least one water-miscible organic solvent should be mixed at a ratio by weight of from 20:80 to 80:20, for example from 30:70 to 70:30. In an additional statement of preference, which is not intended to be mutually exclusive of those ratios mentioned above, it is preferred that the water-miscible organic solvent or the mixture of water-miscible organic solvents included in this part are selected and added to water in an amount sufficient to reduce the surface tension of the water/solvent combination to less than 64 dynes/cm at room temperature.

The at least one water-miscible organic solvent of the present invention may be selected from the group consisting of: C₁₋₆ alkanols such as methanol, ethanol, n-propanol, isopropanol, n-butanol, sec-butanol, tert-butanol, n-pentanol; cyclopentanol; cyclohexanol; diols, particularly diols having from 2 to 12 carbon atoms such as ethylene glycol, propylene glycol, butylene glycol, 1,5-pentanediol, pentylene glycol, hexylene glycol but also including thiodiglycol and oligo- and poly-alkyleneglycols, such as diethylene glycol, triethylene glycol, dipropylene glycol, polyethylene glycol and polypropylene glycol; triols such as 1,2,6-hexanetriol; ketones and ketone-alcohols, such as acetone, methyl ethyl ketone, 2-pentanone, 3-pentanone, methyl isobutyl ketone, cyclohexanone and diacetone alcohol; tetrahydrofuran; dioxane; mono-C₁₋₄-alkyl ethers of diols having from 2 to 12 carbon atoms, such as ethylene glycol mono-(C₁-C₄)-alkyl ethers, propylene glycol mono-(C₁-C₄)alkyl ethers and in particular ethylene glycol monomethyl ether, ethylene glycol monobutyl ether, propylene glycol monomethyl ether, propylene glycol monopropyl ether, propylene glycol monobutyl ether; diethylene glycol mono-(C₁-C₄)alkyl ethers, such as diethylene glycol monomethyl ether and diethylene monobutyl ether; dipropylene glycol mono-(C₁-C₄)alkyl ethers, such as dipropylene glycol N-propyl ether, dipropylene glycol monopropyl ether and dipropylene glycol monobutyl; propylene glycol phenyl ether; linear amides, such as N,N-dimethylformamide and N.N-dimethylacetamide; cyclic amides such as 2-pyrrolidone, N-methyl-2-pyrrolidone, N-ethyl-2-pyrrolidone, caprolactam and 1,3-dimethylimidazolidone; sugar esters such as dimethyl isosorbide; cyclic esters such as caprolactone; and, sulfoxides, such as dimethyl sulfoxide and sulfolane. Said at least one water-miscible solvent is preferably selected from the acetone, diacetone alcohol, isopropyl alcohol and mixtures thereof.

The presence of water-immiscible organic solvents in this formulation is not strictly precluded but it does not represent a preferred embodiment. Rather it is preferred that the formulation is essentially free of water-immiscible organic solvents and as such presents as a single, continuous aqueous phase.

As regards component c) mentioned above, binders or binding agents do represent customary ingredients of aqueous tattoo ink compositions which may be present in an amount up to 5 wt. %, based on the weight of the composition. The binders are non-volatile ingredients which bind particulate colorants to each other and, in doing so, facilitate the intra-dermal or intra-epidermal introduction of the ink using needles and like injection means. Exemplary binders, which may be used alone or in combination, include but are not limited to: polyvinylpyrollidone (PVP), in particular polyvinylpyrollidone having a weight average molecular weight of from 1 to 3000 kDa; polyethylene glycols (PEG), in particular polyethylene glycols having a weight average molecular weight of from 0.2 to 6 kDa; polypropylene glycol (PPG) in particular polypropylene glycols having a weight average molecular weight of from 0.2 to 6 kDa; PEG/PPG-block copolymers, in particular PEG/PPG block copolymers having a weight average molecular weight of from 5 to 15 kDa; PEG/PPG-random copolymers; and, Shellac resin.

The aqueous tattoo ink compositions will typically comprise up to 5 wt. %, for example up to 3 wt. %, based on the weight of the composition, of surfactant. A wide variety of surfactants may be used herein—both for emulsification of the dispersed phase and to provide acceptable spreading of the applied composition—but desirable surfactants will be selected from the group consisting of: anionic surfactants; nonionic surfactants; amphoteric surfactants; non-lathering surfactants; emulsifiers; and, mixtures thereof. The disclosure regarding surfactants provided for Exemplary Embodiment 1 is considered applicable and is incorporated herein by reference.

Examples of pH adjusters: mineral acids such as phosphoric acid; hydroxycarboxylic acids such as lactic acid, glycolic acid, citric acid, 3-hydroxybutyric acid, 4-hydroxybutyric acid, 2-hydroxybutanedioic acid, 2,3-dihydroxybutanedioic acid, 3-hydroxyvaleric acid, 5-hydroxyvaleric acid, 6-hydroxycaproic acid and the alkali metal or alkaline earth metal salts thereof; ammonia; alkali metal hydroxides; alkali metal carbonates; alkali metal bicarbonates; alkaline earth metal hydroxides; alkaline earth metal carbonates; and, alkaline earth metal bicarbonates.

The formulation of this embodiment may comprise from 0 to 5 wt. %, for example from 0 to 3 wt. %, based on the weight of the composition, of one or more thickening agents. Said thickening agents may be selected from: i) carboxylic acid polymers, such as those members of the CARBOPOL®900 series available from B.F. Goodrich; ii) acrylates/C₁₀-C₃₀ alkyl acrylate crosspolymers, such as CARBOPOL®1342, CARBOPOL®1382, PEMULEN® TR-1 and PEMULEN® TR-2 available from B.F. Goodrich; iii) crosslinked polyacrylate polymers, which may be cationic or nonionic polymer; iv) polyacrylamide polymers, in particular nonionic polyacrylamide polymers; iv) multi-block copolymers of acrylamides and substituted acrylamides with acrylic acids and substituted acrylic acids; v) polysaccharides including scleroglucans, cellulose and cellulose derivatives, such as carboxymethylcellulose and alkyl hydroxyalkyl cellulose ethers; vi) modified starches; vii) gums such as xantham gum, acacia gum, alginin, sodium alginate, locust bean gum and guar gum; viii) proteins such as collagen, albumen and gelatin; and, mixtures thereof.

As is known in the art, humectants, or moisturizing agents, are cosmetic ingredients with water binding properties that are capable of retaining large amounts of water relative to their weight. Humectants are usually more soluble in water than in oil. Instructive references on suitable humectants for use in the present formulation include WO98/22085, WO98/18444 and WO97/01326. And exemplary humectants for use herein include: amino acids; collagen amino acids or peptides; keratin amino acids; silk amino acids; urea; glycosaminoglycans; N-acetyl glucosamine; glycerin; polyethylene glycol ethers of glycerin; and, alkali metal salts of hyaluronic acid, acetyl hyaluronic acid, aspartic acid, glucuronic acid and glutamic acid.

The formulation of this embodiment may comprise from 0 to 5 wt. %, preferably from 0 to 2 wt. %, based on the weight of the composition, of at least one antimicrobial agent. It is preferred that the or each antimicrobial agent included in the composition is a crystalline particulate that is insoluble in water. Exemplary antimicrobial agents include but are not limited to: sulfur; piroctone olamine; selenium sulfides, as described in U.S. Pat. Nos. 2,694,668, 3,152,046 and 4,089,945; and, pyridinethione salts, as described in U.S. Pat. Nos. 3,753,196, 4,345,080, 4,323,683, and 4,470,982.

The formulation of this embodiment may comprise from 0 to 2 wt. %, based on the weight of the composition, at least one anti-inflammatory agent. Suitable anti-inflammatory agents, which may be used alone or in combination, include: vitamin F; vitamin E; unsaturated fatty acids; rutin; bioflavonoids; caffeic acid phenethyl ester; sea buckthorn oil; olive oil; jojoba oil; chamomile essential oil; chamomile extract; witch hazel (Hamamelis virginiana) extract; beetroot extract; horseradish extract; dandelion extract; Gamguk extract (Chrysanthemum indicum); Sukbagui extract; betamethasone; dexamethasone; and, mixtures thereof. The aforementioned extracts may be obtained by known methods including hot water or alcoholic extraction processes. A preference for witch hazel extract might be mentioned.

Illustrative antioxidants include vitamin E, vitamin F, vitamin C, rutin, resveratrol, carnosic acid, chitosan, flavonoids, gallates, anthocyanins and carotenoids. Illustrative antiseptics include captan, chlorhexidine, hexachlorophene, triclosan, triacetin and mixtures thereof.

In certain embodiments, the formulation of this embodiment may comprise up to 1 wt. %, based on the weight of the composition, of at least one local anesthetic. As is known in the art, local anesthetics reduce the excitability of sensitive nerve fibers, block the influx of sodium ions through interaction with a binding site on the inside of the membrane of the sodium ion channel and thus prevent the development of action potentials necessary for conduction of excitation. Herein preferred local anesthetics are selected from the group consisting of lidocaine, mepivacaine, prilocaine, articaine, bupivacaine, dibucaine, ropivacaine, etidocaine, dyclonin, procaine, benzocaine, 2-chloroprocaine, oxybuprocaine, tetracaine, fomocaine, etidocaine, pramocaine, Levobupivacaine, oxyprocain, hexylcaine, dibucaine, piperocaine, butamben, butamben picrate, dimethisoquin hydrochloride, diperodone, dyclonine, ketamine, p-butylaminobenzoic acid, pramoxine and their pharmaceutically acceptable salts and mixtures thereof. A preference for lidocaine and benzocaine may be mentioned: this preference is intended to encompass the pharmaceutically acceptable salts of these compounds.

The tattoo ink compositions of the present disclosure may be applied by any conventional technique. As will be recognized by the skilled artisan, the application of the tattoo may be preceded by preparation of the skin by cleaning to remove surface contaminants and/or the application of an anesthetic to provide local and temporary pain relief. The pre-application of a local anesthetic may compliment any anesthetic included within the ink composition itself, as described above.

An intra-epidermal tattoo may be regarded as a “semi-permanent colorant” in that the colorant is disposed within the epidermis but does not diffuse out of the skin and cannot be removed therefrom skin without either the physical disruption or the natural desquamation of the skin. The intra-epidermal tattoo may not, in particular, be washed off under the action or water, soap, alcoholic solvents or combinations thereof.

Epidermal administration of the compositions of the present disclosure may be effected by, for example: needle; microneedle; jet injection; thermal microporation; electroporation; sonoporation; or, combinations thereof. Moreover, the locus of epidermal administration may be to one or more of the stratum corneum, stratum lucidum, stratum granulosum, stratum spinosum and stratum basale.

Without intention to limit the present disclosure, it is considered that techniques for intra-dermal application should be characterized by the introduction of the ink into the dermis under capillary action using a needle which possesses at least three fine points, which points move backwards and forward axially at a frequency of from 50 to 5000 min⁻¹. Under such a mode of introduction, macrophages are released within the dermis as a defensive reaction: given, however, that the ink particulates are much larger than the macrophages, the consequent effect of the ink particulates being surrounded by the phagocytic cells is to fix the ink particulates within the dermis. Under such a mode of introduction, it is also established that a fraction of the ink particulates will be taken up by fibroblasts and a further fraction will be retained within the extracellular matrix of collagen fibers in the dermis. Any ink deposited within the epidermis will however be lost given that epidermal cells are shed both naturally and in a healing response to the injury caused by the needle.

Exemplary tattoo needles for intra-dermal applications include but are not limited to those described in: EP 2454966 A1; US 2007/0038181 A (Melamud); US 2004/0186501 A (Kuei); U.S. Pat. No. 8,764,784 (Crockett); U.S. Design Patent No. 866950 S1 (Schubert); and, U.S. Design Patent No. 888240 S1 (Importla).

Whilst tattoos are intended to be permanent, it is recognized that persons do often seek to remove them from their bodies. The use of melanoidins as pigments is considered to present the benefit that these compounds can be degraded through the targeted action of enzymes and chemicals, such as hydrogen peroxide and ozone, introduced into the dermis or epidermis. The degradation of the melanoidins renders the pigment “removable”.

### Exemplary Embodiment 3: Solid Cosmetic Compositions for Use as Eye Make-Up

In an important embodiment of the present disclosure there is provided a solid cosmetic composition, said composition comprising:
a) at least one colorant, wherein said at least one colorant comprises melanoidins, said melanoidins having a weight average molecular weight of from 200 daltons to 300 kDa, preferably from 1 to 300 kDa and more preferably from 1 to 100 kDa, for example from 10 to 100 kDa;
b) at least one structurant; and,
c) at least one thickener.

It is proposed that solid cosmetic compositions as here defined above may have particular utility as facial make-up, for example as eye make-up. The melanoidins present in the colorant component a) are preferably obtained from the extractive process as defined herein above and in the appended claims. It is noted that the colorant component a) may comprise, consist essentially of or consist of said melanoidins depending on the color effect to be obtained.

In a preferred embodiment, the solid cosmetic composition of the present invention comprises, based on the weight of the composition:
from 2 to 30 wt. % of a) at least one colorant, wherein said at least one colorant comprises melanoidins, said melanoidins having a weight average molecular weight of from 200 daltons to 300 kDa, preferably from 1 to 300 kDa and more preferably from 1 to 100 kDa, for example from 10 to 100 kDa;
from 4 to 50 wt. % of b) at least one structurant;
from 4 to 50 wt. % of c) at least one thickener; and,
from 0 to 90 wt. % d) at least one auxiliary selected from the group consisting of: carriers; stabilizers; surfactants; and, preservatives.

It will be noted that component d) presents categorizations based on the benefit provided by the agent or the agent's postulated mode of action. This categorization is made for convenience: a given chemical ingredient might provide more than one benefit or operate via more than one mode of action.

The present solid cosmetic composition comprises from 4 to 50 wt. %, for example from 5 to 40 wt. % or from 5 to 30 wt. %, based on the weight of the composition, of one or more structurants.

Without intention to limit the present invention, said structurants having utility in the present invention should comprise or consist of waxes have a softening point of from 50 to 150° C. and may include one or more of: i) polyethylene having a number average molecular weight (Mn) from 500 to 7500; ii) petroleum waxes, such as paraffin wax, ozokerite wax, ceresin wax, ader wax, earth wax and microcrystalline wax; iii) synthetic waxes made by polymerizing carbon monoxide and hydrogen, such as Fischer-Tropsch wax; iv) polyolefin waxes; v) hydrogenated animal, fish or vegetable oils; and, vi) plant and animal derived waxes, such as beeswax, carnauba wax, candelilla wax, spermeceti wax and baysberry wax.

The solid cosmetic composition of this embodiment may comprise from 4 to 50 wt. %, for example from 5 to 40 wt. % or from 5 to 30 wt. %, based on the weight of the composition, of one or more thickening agents. Said thickening agents may be selected from: i) carboxylic acid polymers, such as those members of the CARBOPOL®900 series available from B.F. Goodrich; ii) acrylates/C₁₀-C₃₀ alkyl acrylate crosspolymers, such as CARBOPOL®1342, CARBOPOL®1382, PEMULEN® TR-1 and PEMULEN® TR-2 available from B.F. Goodrich; iii) crosslinked polyacrylate polymers, which may be cationic or nonionic polymer; iv) polyacrylamide polymers, in particular nonionic polyacrylamide polymers; iv) multi-block copolymers of acrylamides and substituted acrylamides with acrylic acids and substituted acrylic acids; v) polysaccharides including scleroglucans, cellulose and cellulose derivatives, such as carboxymethylcellulose and alkyl hydroxyalkyl cellulose ethers; vi) modified starches; vii) gums such as xantham gum, acacia gum, alginin, sodium alginate, locust bean gum and guar gum; viii) proteins such as collagen, albumen and gelatin; and, mixtures thereof.

The solid cosmetic composition may comprise up to 50 wt. % of carriers which have the functionality of leaving behind, upon application, a film comprising the melanoidin component as the colorant. Exemplary carriers include: water; water-miscible solvents; water-immiscible solvents; volatile silicones, as described in Todd et al., “Volatile Silicone Fluids for Cosmetics”, Cosmetics and Toiletries, 91:27-32 (1976); non-volatile organic fluids; and, non-volatile silicone fluids. The term “volatile silicone” refers to those silicone materials that have measurable vapor pressure under ambient conditions: cyclomethicone is an important example.

For completeness, non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv™), dipropylene glycol dibenzoate, PPG-15 stearyl ether benzoate and blends thereof (e.g. Finsolv TPP), neopentyl glycol diheptanoate (e.g. Lexfeel 7 supplied by Inolex), octyldodecanol, isostearyl isostearate, octododecyl benzoate, isostearyl lactate, isostearyl palmitate, isononyl/isononoate, isoeicosane, octyldodecyl neopentanate, hydrogenated polyisobutane, and isobutyl stearate. U.S. Pat. No. 6,013,248 (Luebbe et al.) and U.S. Pat. No. 5,968,489 (Swaile et al) are considered to provide instructive references in this context.

The solid cosmetic compositions will typically comprise up to 5 wt. %, for example up to 3 wt. %, based on the weight of the composition, of surfactant. A wide variety of surfactants may be used herein—both for emulsification of the dispersed phase and to provide acceptable spreading of the applied composition—but desirable surfactants will be selected from the group consisting of: anionic surfactants; nonionic surfactants; amphoteric surfactants; non-lathering surfactants; emulsifiers; and, mixtures thereof. The disclosure regarding surfactants provided for Exemplary Embodiment 1 is considered applicable and is incorporated herein by reference.

A preservative may also be present in the solid composition. Examples thereof include: phenoxy ethanol; C₁-C₆ alkyl parabens; imidazolinyl urea; dimethydimethoyl hydantoin; N-(3-chloroallyl) hexaminium chloride; cetrimonium bromide; trisodium ethylene diamine tetraacetic acid; and, butylated hydroxyanisole (BHA). When present, the preservative will typically be added in an amount up to 0.5 wt. %, for instance from 0.01 to 0.1 wt. %, based on the weight of the composition. However, given the preservative functionality of melanoidins, it is also envisaged that said solid cosmetic compositions may be substantially free of preservatives.

The solid cosmetic compositions of the present invention can be formulated as any known or otherwise effective product form for providing topical application of the melanoidin active to the desired area of the skin. Non-limiting examples of such product forms include cakes, sticks, pencils and roll-ons, subject to the proviso that the selected form contains all the essential elements as defined herein. The solid compositions are generally stored in and dispensed from a suitable package or applicator device which should be closeable to prevent loss of any constituent volatile compounds prior to and between applications.

In view of the foregoing description and exemplary embodiments, it will be apparent to those skilled in the art that equivalent modifications thereof can be made without departing from the scope of the claims.

1. A cosmetic composition comprising melanoidins having a weight average molecular weight (Mw) of from 10 kDa to 300 kDa; and
said cosmetic composition is substantially free of caffeine.

2. The cosmetic composition according to claim 1, wherein said cosmetic composition is a non-powdered solid cosmetic composition, a compact powder cosmetic composition or an aqueous liquid cosmetic composition.

3. An aqueous hair dyeing composition comprising:
a) melanoidins having a weight average molecular weight of from 200 daltons to 300 kDa; and,
b) at least one auxiliary agent selected from the group consisting of: wetting agents; swelling agents; penetrants; pH regulators; surfactants; thickeners; antimicrobial agents; and, perfumes, and
said hair dyeing composition is substantially free of caffeine.

4. The aqueous hair dyeing composition according to claim 3 comprising, based on a total weight of the hair dyeing composition:
from 5 to 20 wt. % of said melanoidins;
from 5 to 40 wt. % of said at least one auxiliary agent; and,
from 40 to 90 wt. % of water.

5. An aqueous tattoo ink composition, comprising:
a) at least one colorant comprising melanoidins having a weight average molecular weight of from 200 daltons to 300 kDa;
optionally b) at least one co-solvent; and,
c) at least one auxiliary selected from the group consisting of: binders; thickeners; pH regulators; surfactants; humectants; antimicrobial agents; anti-inflammatory agents; anti-oxidants; antiseptics; and, local anesthetics, and
wherein said tattoo ink composition is substantially free of caffeine.

6. The aqueous tattoo ink composition according to claim 5 comprising, based on a total weight of the tattoo ink composition:
from 10 to 40 wt. % of water;
from 30 to 60 wt. % of said at least one colorant;
from 0 to 15 wt. % of said co-solvent; and,
from 5 to 15 wt. % of said at least one auxiliary.

7. A solid cosmetic composition comprising:
a) at least one colorant comprising melanoidins having a weight average molecular weight of from 200 daltons to 300 kDa;
b) at least one structurant; and,
c) at least one thickener.

8. The solid cosmetic composition according to claim 7 comprising, based on a total weight of the solid cosmetic composition:
from 2 to 30 wt. % of said at least one colorant;
from 4 to 50 wt. % of said at least one structurant;
from 4 to 50 wt. % of said at least one thickener; and,
from 0 to 90 wt. % of at least one auxiliary selected from the group consisting of: carriers; stabilizers; surfactants; and, preservatives.

9. The solid cosmetic composition according to claim 7 that is substantially free of caffeine.

10. The cosmetic composition according to claim 1 wherein said melanoidins have a weight average molecular weight (Mw) of from 10 to 100 kDa.

11. The cosmetic composition according to claim 1, wherein said melanoidins have a weight average molecular weight (Mw) of from 10 to 30 kDa or from 50 to 100 kDa.

12. A process for obtaining at least one melanoidin fraction from a foodstuff, said process comprising steps of:
a) treating a particulate foodstuff comprising melanoidins with an aqueous extractant solution having a pH greater than 7 to extract melanoidins as a solute in an aqueous phase;
b) physically separating said aqueous phase from residual treated particulate foodstuff;
c) fractionating dispersed or dissolved components of said aqueous phase based on molecular weight, wherein said fractionation yields aqueous retentates which are substantially fee from caffeine; and,
d) dehydrating said aqueous retentates to obtain at least one solid melanoidin fraction.

13. The process according to claim 12, wherein the particulate foodstuff is selected from spent coffee grounds and Brewers Spent Grain (BSG).

14. The process according to claim 12, wherein the particulate foodstuff is micronized.

15. The process according to claim 12, wherein said treating step a) comprises treating said particulate foodstuff with an aqueous solution of sodium hydroxide.

16. The process according to claim 12, wherein said treating step a) is performed under centrifugation.

17. The process according to claim 12, wherein said fractionating step c) is performed by: ultra-filtration using molecular weight cut-off membranes; centrifugation; or, size exclusion chromatography.

18. The process according to claim 12, wherein said dehydrating step d) is performed by at least one of: freeze-drying; spray drying; convective drying; radiation drying; and, vacuum drying.

19. The cosmetic composition according to claim 1, wherein said melanoidins are obtained by the process of claim 12.

20. The cosmetic composition according to claim 1, wherein said melanoidins have a weight average molecular weight (Mw) of from 30 kDa to 50 kDa.
